(19) United States
(12) Patent Application Publication
(10) Pub. No.: US 2024/0293589 A1
(43) Pub. Date: Sep. 5, 2024

(54) HYPOCHLOROUS ACID ULTRASOUND PROBE DISINFECTION CHAMBER

(71) Applicant: Disinfection Technology Solutions LLC, Mentor, OH (US)

(72) Inventors: Nick Giarelli, Mentor, OH (US); Mark Piscitelli, Winter Garden, FL (US); Sean Zuckerman, Mentor, OH (US); Russell Donda, Gainesville, FL (US)

(73) Assignee: Disinfection Technology Solutions LLC, Mentor, OH (US)

(21) Appl. No.: 18/663,851

(22) Filed: May 14, 2024

Related U.S. Application Data
(63) Continuation-in-part of application No. 17/686,769, filed on Mar. 4, 2022, now Pat. No. 11,980,696.

(51) Int. Cl. A61L 2/00

(52) U.S. Cl. CPC A61L 2/0088; A61L 2202/122; A61L 2202/14; A61L 2202/15; A61L 2202/24

(57) ABSTRACT

A disinfection chamber for disinfecting probe and probe cord may include a housing, a probe chamber portion defined by the housing, a cord chamber portion defined by the housing, one or more disinfecting portions in operable communication with the probe chamber portion and the cord chamber portion, and a disinfectant composition in operable communication with the one or more disinfecting portions. The one or more disinfecting portions may expel the disinfectant composition within the probe chamber portion and the cord chamber portion to disinfect the probe and at least a portion of the probe cord.

### CROSS-REFERENCE TO RELATED APPLICATIONS

This application is a Continuation-in-Part of and claims priority to U.S. patent application Ser. No. 17/686,769, filed Mar. 4, 2022, which is hereby incorporated herein by reference in its entirety.

### BACKGROUND

There is a need in the field for improvements to conventional disinfection systems for medical probes to make the systems more safe, cost-effective, convenient, and widely available.

### SUMMARY OF THE INVENTION

The present disclosure may provide a disinfection chamber for disinfecting a probe and at least a portion of a probe cord. The probe, such as, for example, an ultrasound probe or transesophageal probe, may be enclosed within a probe chamber portion and at least a portion of the cord may be enclosed within a cord chamber portion. The disinfection chamber may expel a safe disinfectant composition (e.g., a disinfectant composition that is non-toxic to humans) within the enclosed probe chamber portion and the enclosed cord chamber portion to disinfect the probe and the cord, respectively. As such, the disinfection chamber may be used for disinfecting or sterilizing probes and probe cords thereby eliminating all microorganisms, or a suitable level of microorganisms, on the probes and probe cords. The present disclosure may provide one or more disinfection chambers for disinfecting one or more medical devices other than probes and cords. The present disclosure may provide one or more disinfection chambers for disinfecting one or more reusable medical devices.

This is beneficial compared to conventional disinfection systems as conventional disinfection systems may use toxic or unsafe disinfectant compositions and only disinfect a probe leaving the probe cord to be disinfected manually, if at all.

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

### BRIEF DESCRIPTION OF THE DRAWINGS

FIG. 1 illustrates a perspective view of an exemplary disinfection chamber in accordance with one aspect of the present disclosure.

FIG. 2 illustrates a rear perspective view of the exemplary disinfection chamber.

FIG. 3 illustrates a top plan view of the exemplary disinfection chamber.

FIG. 4 illustrates a cross section view looking in the direction of line 4-4 of FIG. 3.

FIG. 5 is an isolated view of an exemplary cord receiving cavity in accordance with one aspect of the present disclosure.

FIG. 6 is a cross section view looking in the direction of line 6-6 of FIG. 4.

FIG. 7 illustrates an example cord winding pattern in plan view and in elevation view.

FIG. 8 illustrates relative elevations of cord support members on two different arrangements of cord chamber surfaces.

FIG. 9 illustrates an electrostatic nozzle spray function onto an example probe.

FIG. 10 is an elevation view of an exemplary probe receiving cavity and a cord receiving cavity having disinfecting portions on a bottom surface.

FIG. 11 is similar to FIG. 10 showing various arrangements of disinfecting portions.

FIG. 12 illustrates a probe closure door having a generally flat face cooperating with the probe receiving cavity.

FIG. 13 is similar to FIG. 3 showing a sensor attached to a disinfecting solution container.

FIG. 14 illustrates an exemplary radio-frequency identification system for use with the disinfection chamber.

FIG. 15 illustrates an exemplary location of sensors for a cord-based interlock.

FIG. 16 illustrates an exemplary arrangement of a transesophageal echocardiogram probe and a probe whip within the probe receiving cavity.

FIG. 17 illustrates an exemplary disinfection chamber having two passageways between the probe receiving cavity and the cord receiving cavity.

FIG. 18 illustrates an exemplary arrangement of the transesophageal echocardiogram probe secured in the cord receiving cavity and the probe whip secured in the probe receiving cavity.

FIG. 19 is a schematic illustration showing relative heights of cord support members and the probe secured within the cord receiving cavity.

FIG. 20 illustrates an exemplary disinfection chamber where the probe receiving cavity is not in operational cooperation with the cord receiving cavity to disinfect two separate medical devices.

FIG. 21 illustrates a flow diagram for an exemplary method for disinfecting a probe and a probe cord.

### DETAILED DESCRIPTION

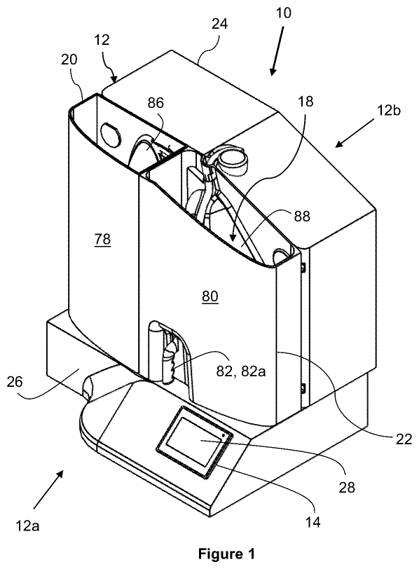

FIG. 1 through FIG. 6 illustrate an exemplary disinfection chamber 10 for disinfecting a probe 1 and its associated probe cord 3. The probe 1 and its associated cord 3 may be any type of probe having any type of cord, such as, for example, an ultrasound probe having an associated ultrasound probe cord.

The disinfection chamber 10 may include a housing 12, a controller 14, and a waste removal assembly 16. The housing 12 may include a front 12a, a rear 12b, a top 12c, a bottom 12d, a first side 12e, and a second side 12f. The front 12a and the rear 12b may define a transverse direction therebetween. The top 12c and the bottom 12d may define a vertical direction therebetween. The first side 12e and the second side 12f may define a longitudinal direction therebetween. The housing 12 may further include a probe chamber portion 20, a cord chamber portion 22, a rear portion 24, and a base 26.

The controller 14 may be provided within the base 26 of the housing 12 and may include a user interface 28. An exemplary controller 14 may be a combination of a human machine interface and a programmable logic controller (PLC) that allows a user to program and/or operate the disinfection chamber as more fully described below.

The probe chamber portion 20 may include a probe chamber surface 30 and a securing mechanism 32. The probe chamber surface 30 may define a recessed probe receiving cavity 34 and a recessed probe transition cavity 36. The securing mechanism 32 may be a clip that releasably secures the probe 1 within the probe receiving cavity 34.

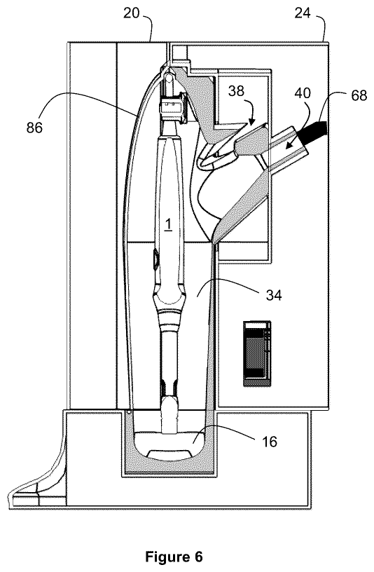

The probe receiving cavity 34 may be in operable communication with the probe transition cavity 36 and the probe receiving cavity 34 may transition to the probe transition cavity 36 at a probe transition point 37. The probe receiving cavity 34 may releasably receive the probe 1 and the probe transition cavity 36 may releasably receive a portion of the cord 3. The probe receiving cavity 34 may define a first aperture 38 and a second aperture 40 extending therethrough (FIG. 6). The probe receiving cavity 34 may take on a substantially inverted rounded V-shape and the probe transition cavity 36 may be provided proximate the top 12c of the disinfection chamber 10 and may extend from the probe receiving cavity 34 in a generally longitudinal direction toward the first side 12e of the disinfection chamber 10.

The cord chamber portion 22 may include a cord chamber surface 42 and one or more cord support members 44. The cord chamber surface 42 may define a recessed cord receiving cavity 48 and a recessed cord transition cavity 50. The cord receiving cavity 48 may be in operable communication with the cord transition cavity 50 and the cord receiving cavity 48 may transition to the cord transition cavity 50 at a cord transition point 51. The cord receiving cavity 48 and the cord transition cavity 50 may each releasably receive a portion of the cord 3. The cord receiving cavity 48 may define a first aperture 52, a second aperture 54, and a third aperture 56. The cord receiving cavity 48 may take on a substantially rounded diamond shape and may include a first curve portion 58, a second curve portion 60, a third curve portion 62, and a fourth curve portion 64. The first curve portion 58 and the second curve portion 60 may be coplanar with one another and the third curve portion 62 and the fourth curve portion 64 may be coplanar with one another.

A first axis X1 may extend through an approximate midpoint of the first curve portion 58 and the second curve portion 60 and a second axis X2 may extend through an approximate midpoint of the third curve portion 62 and the fourth curve portion 64. The first axis X1 and the second axis X2 may intersect with one another at a center point 66 of the substantially diamond shaped cord receiving cavity 48.

The one or more cord support members 44 may be elongated members engaged with the cord receiving cavity 48 such that the one or more cord support members 44 extend in a transverse direction toward the front 12a of the housing 12. In some implementations, the one or more cord support members 44 may include a first cord support member 44a, a second cord support member 44b, a third cord support member 44c, a fourth cord support member 44d, a fifth cord support member 44e, a sixth cord support member 44f, a seventh cord support member 44g, an eighth cord support member 44h, a ninth cord support member 44i, and a tenth cord support member 44j arranged in a particular configuration. For example, the first cord support member 44a may be positioned proximate the cord transition point 51 and between the first aperture 52, the second aperture 54, and the third aperture 56. The remaining nine cord support members 44b, 44c, 44d, 44e, 44f, 44g, 44h, 44i, and 44j may be positioned along the along the first axis X1 and the second axis X2.

In particular, the second cord support member 44b and the sixth cord support member 44f may be positioned along the second axis X1 such that the second cord support member 44b is spaced a distance from the sixth cord support member 44f with the second cord support member 44b being closer to the fourth curve portion 64, the third cord support member 44c, the seventh cord support member 44g, and the tenth cord support member 44j may be positioned along the first axis X1 such that the third cord support member 44c, the seventh cord support member 44d and the tenth cord support member 44j are spaced a distance from one another with the third cord support member 44c being closest to the second curve portion 60 and the seventh cord support member 44g being closer to the second curve portion 60 than the tenth cord support member 44j, the fourth cord support member 44d and the eighth cord support member 44h may be positioned along the second axis X2 such that the fourth cord support member 44d is spaced a distance from the eighth cord support member 44h with the fourth cord support member 44d being closer to the third curve portion 62, and the fifth cord support member 44e and the ninth cord support member 44i may be positioned along the first axis X1 such that the fifth cord support member 44e is spaced a distance from the ninth cord support member 44i with the fifth cord support member 44e being closer to the first curve portion 58.

The first cord support member 44a, the second cord support member 44b, the third cord support member 44c, the fourth cord support member 44d, and the fifth cord support member 44e may each have a substantially similar first length, the sixth cord support member 44f, the seventh cord support member 44g, the eighth cord support member 44h, and the ninth cord support member 44i may each have a substantially similar second length, and the tenth cord support member 44j may have a third length. The first length may be less than the second length and the third length and the second length may be less than the third length.

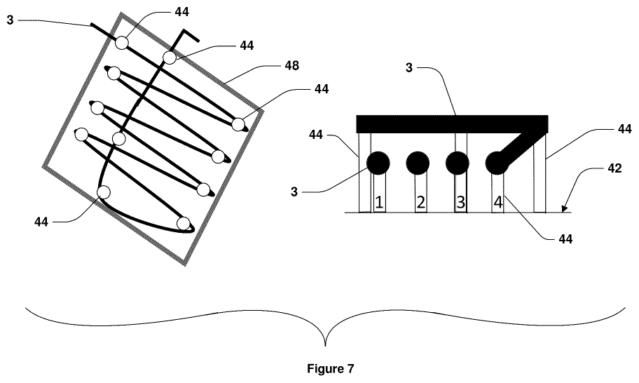

Referring to FIG. 7, other cord support member 44 arrangements and heights can be provided to provide any number of cord winding patterns in any number of cord receiving cavity geometries. While not meant to be limiting, arrangements can include winding the cord 3 in a back-and-forth pattern alternating from one cord support member 44 on one side of the cord receiving cavity 48 to another cord support member 44 on an opposing side of the cord receiving cavity 48 to resemble a stalactite, stalagmite, or sawtooth-like configuration. As previously discussed, the cord support members 44 can extend from the cord chamber surface 42 to various heights to expose a maximum amount of surface area of the cord 3 for disinfection. The various height cord support members 44 can also reduce the number of tangles or knots of the cord 3. As shown in FIG. 7, portions of the cord 3 entering and exiting the cord receiving cavity 48 can be supported at a higher elevation or distance from the cord chamber surface 42 compared to a middle portion of the cord 3 supported by the cord support members 44 labeled 1, 2, 3, and 4. The cord support members 44 can include various structures including hooks or stanchions to support the cord 3 and enable the cord 3 to zig-zag back and forth within the cord receiving cavity 48 or the disinfection chamber 10.

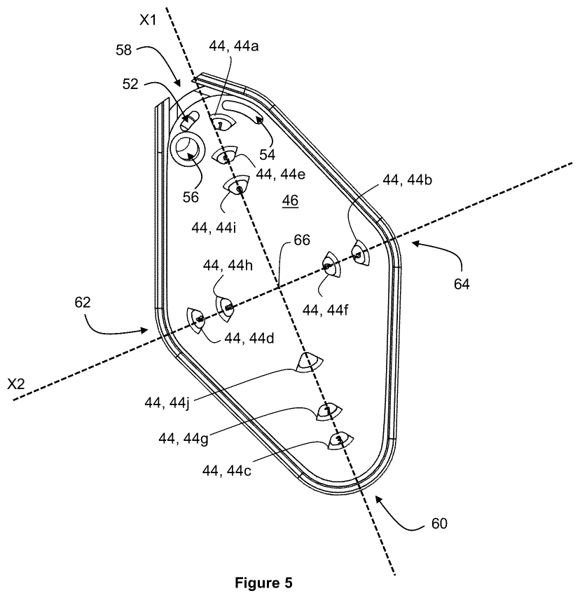

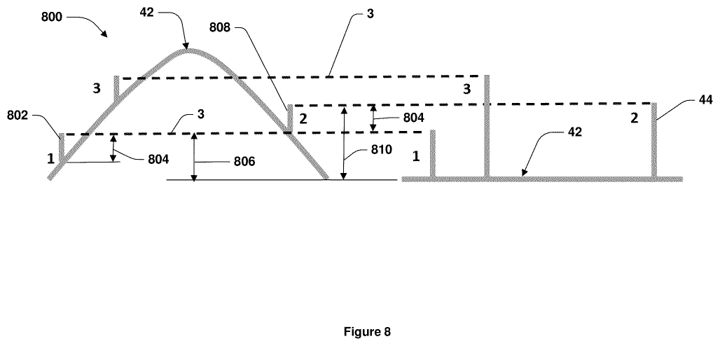

Referring to FIG. 8, two example cord chamber surfaces 42 are shown side-by-side in A and B for comparison. The arrangement on the right B side corresponds to that of chamber surface 42 of FIG. 5 including cord support members 44 of different heights. As shown on the left A side, in one embodiment, the cord chamber surface 42 can define a convex portion 800 extending toward the cord chamber door (not shown in FIG. 8) such that a first cord support member 802 having a first length 804 is configured to support the cord 3 at a first distance 806 from the cord chamber surface 42. The second cord support member 808 having the first length 804 is configured to support the cord 3 at a second distance 810 from the cord chamber surface 42. As such, the cord receiving cavity 48 can include a domed or arched “floor” to achieve three or more ranges of cord support height using cord support members 44 of the same length or of several quantity of lengths if the several quantity of lengths are fewer than the total number of distances 806, 810, etc. In FIG. 8, the dashed lines are used to compare the distances from support locations of the cord support members 44 to the cord chamber surface 42 of the two A and B floor schemes shown.

The described example cord chamber surface 42 on the left A side of FIG. 8 can minimize a volume of the cord chamber and an amount of the disinfectant consumed per disinfection cycle. In some implementations, the example domed cord chamber surface 42 can cooperate with a complementary profile aspect of the cord chamber door (e.g., the cord chamber door may also include a dome-like surface).

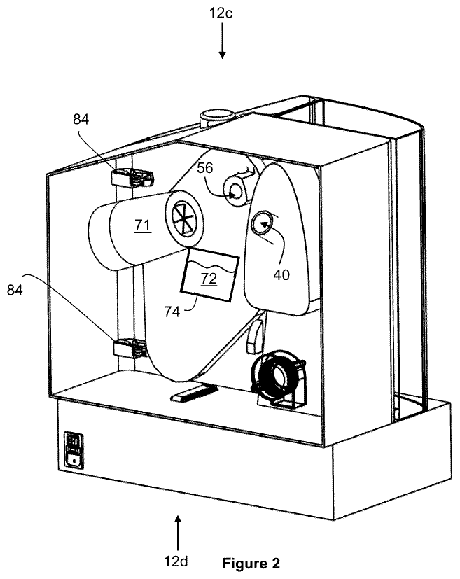

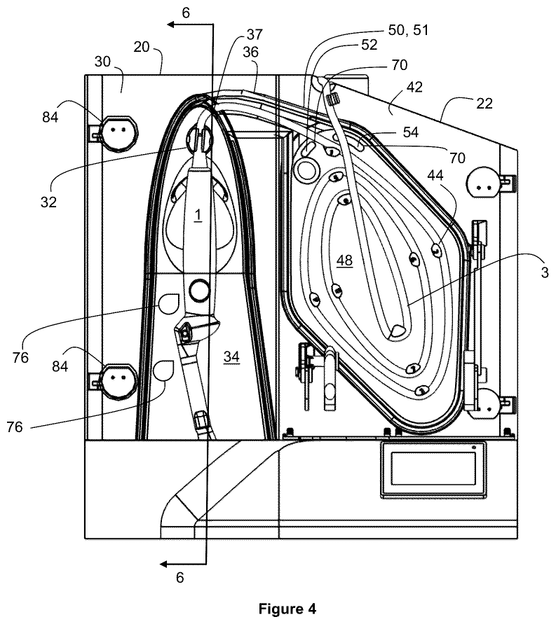

Returning to FIGS. 1-6, the disinfection chamber 10 may further include a probe disinfecting portion 68 (FIG. 6), a cord disinfecting portion 70 (FIG. 4). In some implementations, the probe disinfecting portion 68 and the cord disinfecting portion 70 may be ultrasonic nebulizers, such as, for example, piezoelectric nebulizers. As such, the probe disinfecting portion 68 and the cord disinfecting portion 70 may be in operable communication with a heated blower 71 and a disinfectant composition 72 being stored within a disinfectant composition storage container 74. The probe disinfecting portion 68 may be in fluid communication with the probe chamber portion 20 via the second aperture 40 (FIG. 6) of the probe receiving cavity 34 and the cord disinfecting portion 70 may be in fluid communication with the cord chamber portion 22 via the third aperture 56 (FIG. 2) of the cord receiving cavity 48. The first aperture 38 of the probe receiving cavity 34, the second first aperture 52 of the cord receiving cavity 48, and the second aperture 54 of the cord receiving cavity 48 may serve as vents allowing suitable pressurization of the probe chamber portion 20 and the cord chamber portion 22.

The disinfectant composition 72 may be electrolyzed water, which is produced by electrolyzing water containing dissolved sodium chloride (e.g., tap water). In particular, the reaction products may be a solution of hypochlorous acid (HOCI) and sodium hydroxide (NaOH) and the solution may be used as a disinfectant. Exemplary benefits of using electrolyzed water as a disinfectant solution is that it is non-toxic to humans. In some implementations, a potential of hydrogen (pH) of the disinfectant composition 72 may be between a range of 4.8 and 5.2; however, it is to be understood that the pH of the disinfectant composition 72 may be any suitable pH.

The probe disinfecting portion 68, the cord disinfecting portion 70, the heated blower 71, the disinfectant composition 72, and the disinfectant composition storage container 74 may operate to generate droplets 76 of the disinfectant composition 72 to be expelled within the probe chamber portion 20 (through the second aperture 40) and the cord chamber portion 22 (through the third aperture 56), respectively.

In some implementations, the probe disinfecting portion 68 may expel the droplets 76 in the form of a mist within the probe chamber portion 20 to disinfect the probe 1 and the cord disinfecting portion 70 may expel the droplets 76 in the form of a mist within the cord chamber portion 22 to disinfect the cord 3. The droplets 76 generated by the probe disinfecting portion 68 and the cord disinfecting portion 70 may have a size in a range from twelve microns to forty microns. The droplets 76 generated by the probe disinfecting portion 68 and the droplets 76 generated by the cord disinfecting portion 70 are not necessarily the same size and their size(s) can be varied to suit a particular probe or a particular cord being disinfected. The size(s) of the droplets 76 can be varied for any other variables as well.

As the probe disinfecting portion 68 and the cord disinfecting portion 70 may use vibration to generate and expel the droplets 76, the size of the droplets 76 may be adjusted based, at least in part, on a frequency of the vibration used by the probe disinfecting portion 68 and the cord disinfecting portion 70. Stated otherwise, the size of the droplets 76 may be controlled as desired by changing the frequency of the vibration associated with the probe disinfecting portion 68 and the cord disinfecting portion 70.

While the probe disinfecting portion 68 has been described as being in fluid communication with the probe chamber portion 20 via the second aperture 40 and the cord disinfecting portion 70 as being in fluid communication with the cord receiving cavity 48 via the third aperture 56, it is to be understood that the probe disinfecting portion 68 and the cord disinfecting portion 70 may be provided in any suitable location within the housing 12 to be in fluid communication with the probe chamber portion 20 and the cord chamber portion 22, respectively.

For example, in some implementations, the probe disinfecting portion 68 (shown in FIG. 6) can include a nozzle to introduce the disinfectant 72 (shown in FIG. 2) into the probe receiving cavity 34. Similarly, the cord disinfecting portion 70 (shown in FIG. 4) can include a nozzle to introduce the disinfectant 72 (shown in FIG. 2) into the cord receiving cavity 48. Any suitable nozzle type can be used, including, but not limited to, ultrasonic, electrostatic, vibrating solid surface, vibrating mesh, pressure driven ultrasonic, etc.

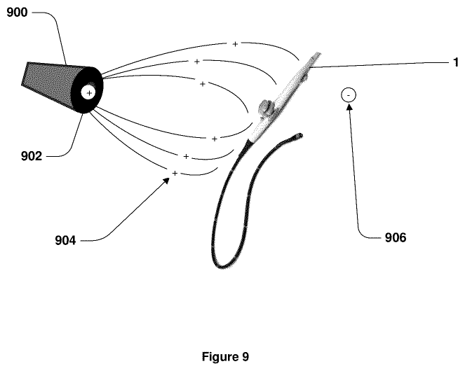

Referring to FIG. 9, in the case of electrostatic nozzles, the nozzle 900 includes a positive diode 902 to impart a positive charge 904 onto the disinfectant 72 being introduced to the probe 1. In some implementations, the probe 1 will be given a negative charge as shown at 906 to increase an attractive force between the positively charged disinfectant 72 and the negatively charged probe 1.

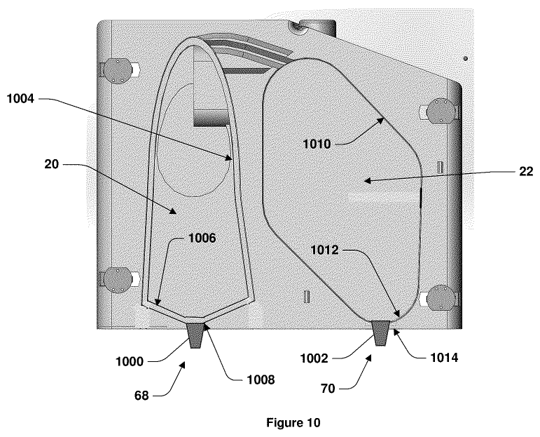

Referring to FIG. 10, the probe disinfecting portion 68 and the cord disinfecting portion 70 can include nozzles located at various other locations than those previously described. In some implementations, the probe disinfecting portion 68 can include a nozzle 1000 located at or near a bottom surface of the probe chamber portion 20. Similarly, the cord disinfecting portion 70 can include a nozzle 1002 located at or near a bottom surface of the cord chamber portion 22. The probe chamber portion 20 can include a probe chamber side wall 1004 that can include a probe chamber bottom wall 1006. The probe chamber bottom wall 1006 defines a bottom wall aperture 1008 configured to place the one or more disinfecting portions 68 in operable communication with the probe receiving cavity 34. Likewise, the cord chamber portion 22 can include a cord chamber side wall 1010 that includes a cord chamber bottom wall 1012. The cord chamber bottom wall 1012 defines a bottom wall aperture 1014 configured to place the one or more disinfecting portions 70 in operable communication with the cord receiving cavity 48.

Locating the nozzles 1000, 1002 at, on, or near the bottom walls 1006, 1108 producing a mist of droplets upward can beneficially eliminate a need for a waste reservoir 16 (shown in FIG. 6). The bottom walls 1006, 1012 can be shaped or configured to direct a quantity of condensate of the disinfectant 72 toward the one or more disinfecting portions 68, 70. In some implementations, the bottom walls 1006, 1012 can be configured to urge or direct any fluid toward the one or more disinfecting portions 68, 70. In other words, the surfaces and apertures can beneficially direct any condensation of disinfectant 72 and/or excess disinfectant 72 back into the nozzle 1000, 1002, or the nozzle reservoir which can help to conserve or to re-use the disinfectant 72.

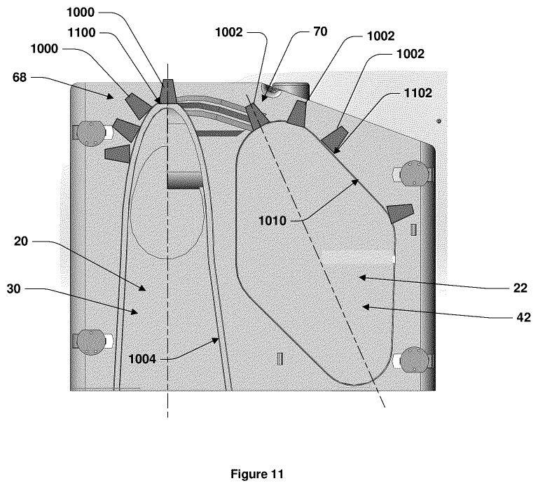

Referring to FIG. 11, the probe disinfecting portion 68 and the cord disinfecting portion 70, can be located at still other locations. In some implementations, the probe chamber side wall 1004 defines a side wall aperture 1100 configured to place the one or more probe disinfecting portions 68 in operable communication with probe chamber portion 20 such that the disinfectant 72 is introduced in a direction parallel to the probe chamber surface 30. The side wall aperture 1100 can also be configured to place the one or more probe disinfecting portions 68 such that the disinfectant 72 is introduced in a direction substantially parallel to the probe chamber surface 30 (e.g., within five degrees of parallel to the probe chamber surface 30).

Additionally, the cord chamber side wall 1010 defines a side wall aperture 1102 configured to place the one or more cord disinfecting portions 70 in operable communication with the cord chamber portion 22 such that the disinfectant 72 is introduced in a direction parallel to the cord chamber surface 42. The side wall aperture 1102 can also be configured to place the one or more cord disinfecting portions 70 such that the disinfectant 72 is introduced in a direction substantially parallel to the cord chamber surface 42 (e.g., within five degrees of parallel to the cord chamber surface 42).

The nozzles 1000, 1002 can introduce the disinfectant 72 in a direction parallel to the main axes of the chambers 20, 22 (i.e. downward into the chambers 20, 22). In some examples, the direction of disinfectant introduction may be straight down (e.g. from a 12 o'clock dial position downward toward the probe 1 or the cord 3). The direction can also be oblique to the main axis (e.g. 10 o'clock or 11 o'clock in the probe chamber portion 20 and 1, 2, or 3 o'clock in the cord chamber portion 22). Any suitable type nozzles 1000, 1002 can be used in this configuration.

It is to be understood that the probe disinfecting portion 68 and the cord disinfecting portion 70 can operate independently from one another. For example, a first nozzle 1000 of the one or more nozzles 1000 in operable communication with the probe receiving cavity 34 is configured to introduce the disinfectant 72 into the probe receiving cavity 34 as a mist of droplets having a first droplet size. A second nozzle 1002 of the one or more nozzles 1002 in operable communication with the cord receiving cavity 48 is configured to introduce the disinfectant 72 into the cord receiving cavity 48 as a mist of droplets having a second droplet size where the first droplet size is different than the second droplet size.

In another example, the first nozzle 1000 in operable communication with the probe receiving cavity 34 is configured to introduce a first volume of the disinfectant 72 into the probe receiving cavity 34 while the second nozzle 1002 in operable communication with the cord receiving cavity 48 is configured to introduce a second volume of the disinfectant 72 into the cord receiving cavity 48 where the first volume is different than the second volume. In some examples, one of the volumes can be equal to zero.

In yet another example, the first nozzle 1000 in operable communication with the probe receiving cavity 34 includes a first nozzle type, and the second nozzle 1002 in operable communication with the cord receiving cavity 48 includes a second nozzle type that is different than the first nozzle type. In a still further example, the first nozzle 1000 can be configured to introduce a first disinfectant into the probe receiving cavity 34 that is different than a second disinfectant introduced into the cord receiving cavity 48 by the second nozzle 1002.

Referring to FIGS. 6, 10, and 11, the probe disinfecting portion 68 and the cord disinfecting portion 70, as noted previously, can include many different types of structures to introduce the disinfectant composition 72 within the probe chamber portion 20 and the cord chamber portion 22. In some implementations, the probe disinfecting portion 68 and the cord disinfecting portion 70 include at least one of a nozzle 1000, 1002 or a perforated surface to introduce the disinfectant composition 72 within the probe chamber portion 20 and the cord chamber portion 22. In some examples, the perforated surface can be a portion of a manifold that is in operable communication with the probe disinfecting portion 68 and the cord disinfecting portion 70, but this example is not meant to be limiting, and any suitable perforated surface can be used with the present disclosure. In some implementations, the perforated surface can be a portion of any of the chamber surfaces 30, 42 the chamber side walls 1004, 1010, or other walls that define the probe chamber portion 20 and the cord chamber portion 22. The perforated surface can be engineered and manufactured to produce droplets of the disinfectant composition 72 that introduce the disinfectant composition 72 within the probe chamber portion 20 and the cord chamber portion 22. The droplets produced by the perforated surface can be larger than the 40 micron diameter size average previously discussed.

Regardless of whether the probe disinfecting portion 68 and the cord disinfecting portion 70 include a nozzle, a perforated surface, or any other suitable structure configured to introduce the disinfectant composition 72 within the probe chamber portion 20 and the cord chamber portion 22, several techniques can be used to urge introduction of the disinfectant composition 72. In some implementations, a fluid can be used to introduce the disinfectant composition 72 within the probe chamber portion 20 and the cord chamber portion 22. For example, a moving gas (e.g., air), can create a force to urge the disinfectant composition 72 through the probe disinfecting portion 68 and the cord disinfecting portion 70 into the probe chamber portion 20 and the cord chamber portion 22. The moving gas can force or drive a substance (e.g., the disinfectant composition 72) out of the nozzle, manifold, or perforated surface to introduce the disinfectant composition 72 within the probe chamber portion 20 and the cord chamber portion 22. For the purposes of this disclosure, a moving gas can include any suitable gas capable of passing from a first side of the disinfecting portions 68, 70 that is outside of the chamber portions 20, 22 to a second side of the disinfecting portions 68, 70 that is inside of at least one chamber portion 20, 22. A moving gas can also include a compressed gas, a gas moving at relatively low speed that is urged into motion by a rotating fan blade, or any number of other gases and structures configured to move the gas.

In some implementations, hydraulic pressure can be used to introduce the disinfectant composition 72 within the probe chamber portion 20 and the cord chamber portion 22. For example, a force acts upon a fluid to create hydraulic pressure, and the resulting hydraulic pressure urges the disinfectant composition 72 through the probe disinfecting portion 68 and the cord disinfecting portion 70 into the probe chamber portion 20 and the cord chamber portion 22. In this example, the disinfectant composition 72 is urged or pushed through the nozzle, manifold, perforated surface, etc., converting hydraulic fluid pressure into kinetic energy (e.g., motion of the disinfectant composition 72). The hydraulic fluid pressure can cause the disinfectant composition 72 to spray from the probe disinfecting portion 68 and the cord disinfecting portion 70 into the probe chamber portion 20 and the cord chamber portion 22.

In still other implementations, the probe disinfecting portion 68 and the cord disinfecting portion 70 can include a vibrating surface. The vibrating surface can be a solid surface or a mesh-like surface used to create a mist or spray of relatively small droplets of the disinfectant composition 72 that then pass from the probe disinfecting portion 68 and the cord disinfecting portion 70 into the probe chamber portion 20 and the cord chamber portion 22. The vibrating surface or vibrating surfaces disrupt the relatively orderly arrangement of portions of the disinfectant composition 72 into fine particles (e.g., droplets) that are then expelled to create a spray or mist introduced into the probe chamber portion 20 and the cord chamber portion 22.

Regardless of the structure type of the probe disinfecting portion 68 and the cord disinfecting portion 70, the structure type can be selected or engineered to take advantage of various physical principles of fluid movement and fluid droplet size to achieve the desired goal of introducing the disinfectant composition 72 within the probe chamber portion 20 and the cord chamber portion 22. Additionally, the structure type can be selected or engineered to take advantage of various physical principles of fluid movement and fluid droplet size to best meet the requirements or standards designed to properly disinfect particular medical instruments or other objects.

Referring back to FIGS. 1-6, the waste removal assembly 16 may include one or more waste reservoirs 16a and one or more waste evacuation devices (not shown) for collecting disinfectant composition 72 waste within the probe chamber portion 20 and the cord chamber portion 22 and evacuating the collected disinfectant composition 72 waste from within the probe chamber portion 20 and from within the cord chamber portion 22.

The disinfection chamber 10 may further include a probe chamber door 78, a cord chamber door 80, and a locking mechanism 82. The probe chamber door 78 may selectively enclose the probe receiving cavity 34 and the probe transition cavity 36 and the cord chamber door 80 may selectively enclose the cord receiving cavity 48 and the cord transition cavity 50.

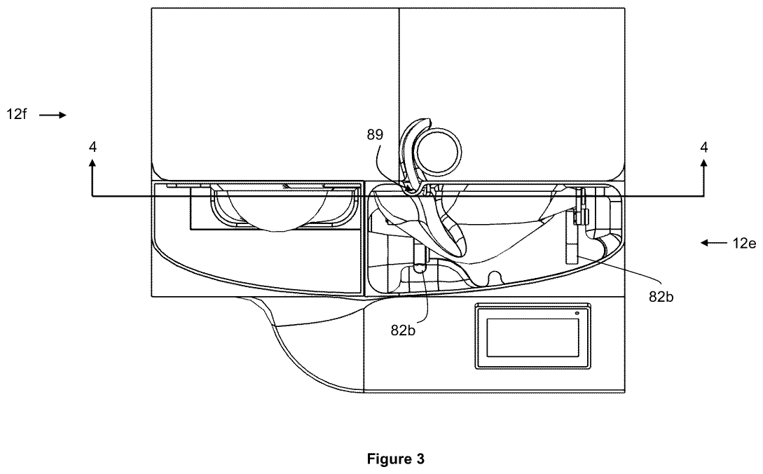

The probe chamber door 78 and the cord chamber door 80 may be operably engaged with the housing via hinges 84. The probe chamber door 78 may define a probe enclosing portion 86 and the cord chamber door 80 may define a cord enclosing portion 88 therein and an exit aperture 89 (FIG. 3).

The exit aperture 89 may be provided proximate the top 12c of the housing 12 and may allow a portion of the cord 3 to pass therethrough. The probe enclosing portion 86 may be complementary in shape to the probe receiving cavity 34 and the probe transition cavity 36 and the cord enclosing portion 88 may be complementary in shape to the cord receiving cavity 48 and the cord transition cavity 50.

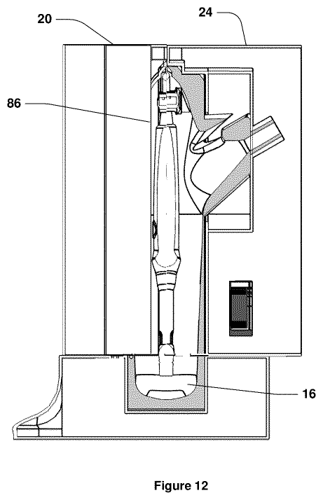

In some implementations, such as that shown in FIG. 12, the probe enclosing portion 86 can be flat, and is not complementary in shape to the probe receiving cavity 34. This flat probe enclosing portion can be used with any of the examples within this disclosure.

Returning to FIGS. 1-6, the probe chamber door 78 may be moveable between an open position and a closed position. As such, the probe chamber door 78 may be moved to the open position to allow access to the probe receiving cavity 34 and the probe transition cavity 36 and may be moved to the closed position to enclose the probe receiving cavity 34 and the probe transition cavity 36. The cord chamber door 80 may be moveable between an open position and a closed position. As such, the cord chamber door 80 may be moved to the open position to allow access to the cord receiving cavity 48 and the cord transition cavity 50 and may be moved to the closed position to enclose the cord receiving cavity 48 and the probe transition cavity 36.

The locking mechanism 82 may include a handle 82a and a linkage assembly 82b. The locking mechanism 82 may be operably engaged with the cord chamber door 80. The locking mechanism 82 may be moveable between a locked position and an unlocked position to lock and unlock the cord chamber door 80.

In some implementations, a first locking mechanism can be attached to the first chamber door (e.g., probe chamber door 78) and a second locking mechanism can be attached to the second chamber door (e.g., cord chamber door 80). The first locking mechanism and the second locking mechanism are configured to operate dependently such that only one of the probe chamber door 78 and the cord chamber door 80 are operable at one time. In some further examples, a single locking mechanism may be used to the same effect.

Use of two locking mechanisms or two latches operated dependently such that only one door and disinfecting device can be handled at a single time can reduce or eliminate cross-contamination between medical devices, cords, whips, and any combination thereof. In some examples, a radio-frequency identification (RFID) system can be used in conjunction with the disinfection chamber. After a user disinfects an object, an associated printer creates a label to track disinfection data (e.g., time, date, operator/user, device serial number, etc.). A user interface (e.g., a touchscreen) can then require the user to verify via an identification badge that a first object (e.g., medical device) is bagged, labeled, and stored before the second locking mechanism or latch will unlock to provide access to the other disinfecting chamber.

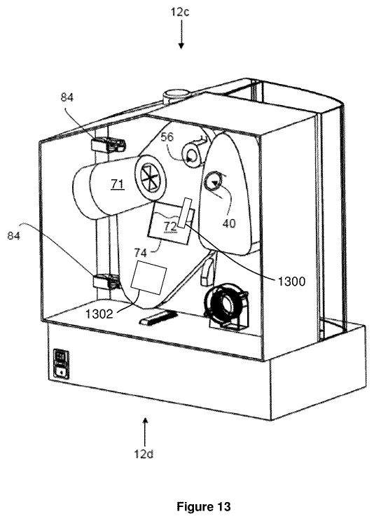

Referring to FIG. 13, the disinfectant storage container 74 can be attached to the housing 12 or contained within the housing 12. A sensor 1300 can be attached to the housing 12 or the disinfectant composition storage container 74. The sensor 1300 can include, but is not limited to, a conductivity sensor or an electrochemical sensor. In the case of the sensor 1300 being a conductivity sensor, the sensor 1300 may be configured to measure a conductivity property of the disinfectant 72. In some implementations, the conductivity measurement of the disinfectant 72 occurs prior to a disinfection cycle start as safety interlock to help ensure an effective disinfection operation. Conductivity thresholds for electrolyzed acid water and other disinfectants that decompose or deactivate can be established to help ensure accurate and effective disinfection without the need for user interpreted chemical indicators (color-based or otherwise).

If disinfectant conductivity is below a minimum threshold for disinfection, the controller will prompt the user via the user interface to change or replace a disinfectant bottle (e.g., the disinfectant storage container 74). The replacement disinfectant within the new disinfectant bottle will be measured for conductivity and the process will be repeated if the measured conductivity of the replacement disinfectant is below a minimum threshold.

In the case of the sensor 1300 being an electrochemical sensor, the sensor 1300 may be configured to measure an electrochemical property of the disinfectant 72. In some examples, the electrochemical measurement of the disinfectant 72 occurs prior to a disinfection cycle start as safety interlock to help ensure an effective disinfection operation. Electrochemical property thresholds for electrolyzed acid water and other disinfectants that decompose or deactivate can be established to help ensure accurate and effective disinfection without the need for user interpreted chemical indicators.

If a disinfectant electrochemical measurement is outside an accepted range for disinfection, the controller will prompt the user via the user interface to change the disinfectant bottle (e.g., the disinfectant storage container 74). The replacement disinfectant will be measured for the electrochemical property and the process will be repeated if the measured electrochemical property of the replacement disinfectant is outside an accepted range for disinfection.

As previously discussed, a moving gas (e.g., compressed air) can create a force to urge the disinfectant composition 72 through the probe disinfecting portion 68 and the cord disinfecting portion 70 into the probe chamber portion 20 and the cord chamber portion 22. In implementations that use this technique, to urge movement of the disinfectant composition 72, the moving gas can also pass into the probe chamber portion 20 and the cord chamber portion 22. Introduction of the moving gas into the chamber portions 20, 22 can cause increased gas pressures within the chamber portions 20, 22 that can be accommodated by various structures and techniques of using those structures.

As shown in FIG. 13, the disinfection chamber 10 can include a flexible structure 1302 configured to accommodate pressure changes within the chamber portions 20, 22. In some implementations, the flexible structure 1302 includes material that is flexible or pliable in order to expand and contract to accommodate the described pressure changes. In some implementations, the flexible structure 1302 includes an elastomeric material, however, any suitable material can be used to construct the flexible structure 1302.

In some implementations, the flexible structure 1302 is formed as a chamber or bladder in operable communication with at least one of the probe chamber portion 20 or the cord chamber portion 22. The probe chamber portion 20 and the cord chamber portion 22 are in operable communication with each other such that the gas pressures within the respective chamber portions 20, 22 will be equal or nearly equal. As such, the flexible structure 1302 in operable communication with only one of the chamber portions 20, 22 can operate to accommodate pressure changes and/or dampen pressure changes within both chamber portions 20, 22. In some implementations, the flexible structure 1302 can be a flexible membrane sheet or sac-like component in operable communication with the chamber portions 20, 22 that can expand and contract. Flexibility of the flexible structure 1302 allows the structure 1302 to adjust in size and/or shape according to the internal pressure changes and directly respond to pressure changes happening within the chamber portions 20, 22. Design of the flexible structure 1302 can enable expansion or inflation upon introduction of a gas into either of the chamber portions 20, 22 and then contract or deflate to urge the gas back into the chamber portions 20, 22 in a controlled manner. In some implementations, the gas will pass through at least one of the apertures in the walls of the chamber portions 20, 22 or through at least one of the opened probe chamber door 78 or the opened cord chamber door 80 after completion of the disinfection operation.

In other implementations, the flexible structure 1302 can include a window-like structure including a flexible or pliable material configured to expand and contract to accommodate and/or dampen pressure changes within both chamber portions 20, 22. The flexible structure 1302 of this example can be formed into one or more walls of either or both chamber portions 20, 22. In still other implementations, the flexible structure 1302 can be a container having dimensions suitable to contain the introduced gas or suitable to accommodate and/or dampen the described pressure changes within the chamber portions 20, 22.

In summary, the flexible structure 1302 can be configured to accommodate potential pressure changes caused by the introduction of gas into the chamber portions 20, 22. For example, introduction of pressurized gas into the chamber portions 20, 22 can cause pressure changes by one or more processes. These processes can include, but are not limited to: adding volume of material within the chamber portions 20, 22; reacting chemically with material present within the chamber portions 20, 22, and changing the temperature within the chamber portions 20, 22. The flexible structure 1302 is configured to absorb or minimize these pressure fluctuations. By expanding or contracting, the flexible structure 1302 can prevent or help prevent sudden increases or decreases in pressure that could potentially damage the disinfection chamber 10 or affect the efficiency of the disinfection chamber 10.

The flexible structure 1302 can help promote effective and repeatable operation of the disinfection chamber 10. The flexible structure 1302 can also help maintain structural integrity and consistent performance of the disinfection chamber 10 even as conditions within the disinfection chamber 10 change.

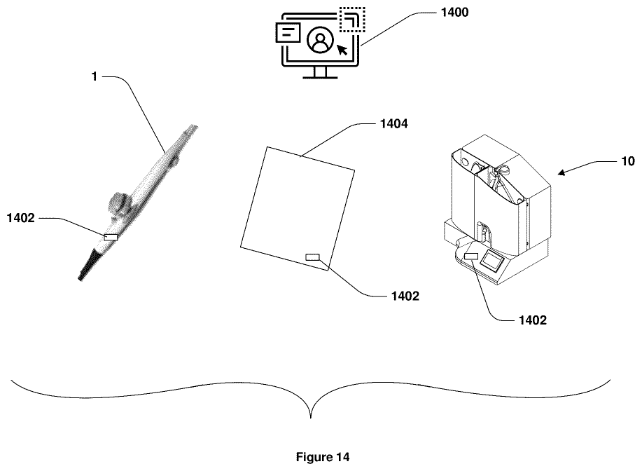

Referring to FIG. 14, an identification system may be used to create a searchable database 1400 including disinfection information relating to the probe 1 or any other object subject to disinfection. The identification system may include any type of contact or contactless identification technologies such as barcodes, QR codes, near field communication (NFC), radio frequency identification (RFID), etc. In the exemplary case of RFID, the RFID system can include an RFID tag 1402 unique to the probe 1 and an RFID tag 1402 unique to a container 1404 of the disinfectant 72 to track lot numbers, shelf life, and other attributes. Through an identification badge or other method (e.g., finger print, retina scan, etc.), a unique identity for a user of the RFID system can be entered. In further examples, an associated RFID reader (not shown) can be configured to communicate with a controller and a programmable logic controller (PLC) associated with the disinfecting chamber.

The described RFID system can create or input data for the searchable database 1400 for each disinfection operation, the input data including a record of a date, a time, a probe identification number, a user identification, a disinfectant lot, and a disinfectant conductivity. In some examples, a label is placed on a bag containing the disinfected probe and is stored until its next use. The database can be available or accessible to download, print, or display in associated software, an application for a smartphone, etc.

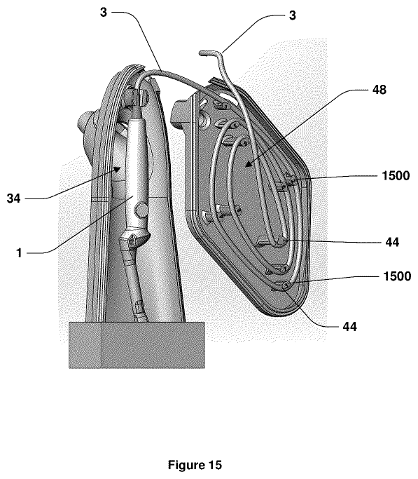

Referring to FIG. 15, the first cord support member 44 includes a sensor 1500 attached to the first cord support member 44. The sensor 1500 is configured to sense contact between the cord 3 and the first cord support member 44. Any suitable combination or number of pairs of sensors 1500 and cord support members 44 can be used with the present disclosure, for example, every cord support member 44 can include a sensor 1500. The sensor 1500 can be in electrical communication with the controller and the PLC of the disinfection chamber 10 to provide a system interlock. The system interlock can be based upon a minimum number of sensors 1500 attached to the cord support members 44 confirming the length of the cord 3 within the cord receiving cavity. This interlock can help ensure that a minimum length of the cord 3 is inside the cord receiving cavity for a disinfection operation. The number of interlocks showing positive contact with the cord 3 can be tailored to a specific length of the cord 3 during a probe RFID tag assignment. The interlock can be further tailored to include some, all, or none of the cord 3 as desired.

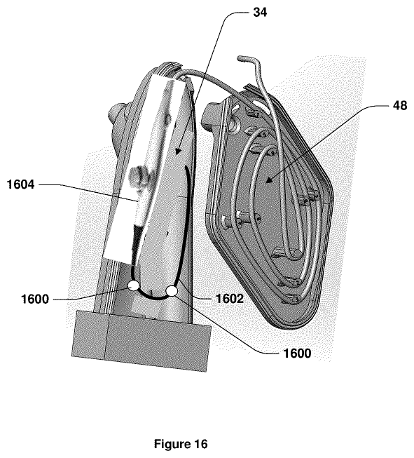

Referring to FIG. 16, a whip support 1600 may be attached to and extending away from the probe chamber surface 30, the whip support 1600 configured to support a probe whip 1602 a distance away from the probe chamber surface 30. In some disinfecting operation examples, the system may be used to disinfect transesophageal echocardiography (TEE) probes. The TEE probe typically includes a control body 1604 and a probe whip 1602 extending away from the control body 1604. As such, the probe receiving cavity 34 is able to hold and secure a TEE probe (control body 1604) and the whip 1602 associated with the TEE probe.

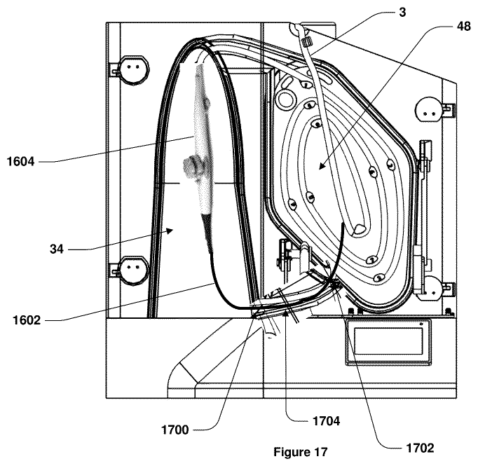

Referring to FIG. 17, the system may include a second probe transition cavity 1700 and a second cord transition cavity 1702. In combination, the second probe transition cavity 1700 and the second cord transition cavity 1702 define a second passageway 1704 between the probe receiving cavity and the cord receiving cavity. This second passageway 1704 or transition path from probe chamber to cord chamber can be located at a bottom area of the probe receiving cavity to pass the TEE probe whip 1602 into the cord receiving cavity. In some examples, the whip 1602 and the cord 3 may be supported in different planes within the cord receiving cavity 48 to avoid contact between one another. In some examples, there can be whip-specific supports 44 in the cord receiving cavity 48.

The second passageway 1704 enables the probe whip 1602 attached to the probe control body 1604 to pass from the probe receiving cavity 34 to the cord receiving cavity 48 such that a portion of the probe whip 1602 is received within the cord receiving cavity 48.

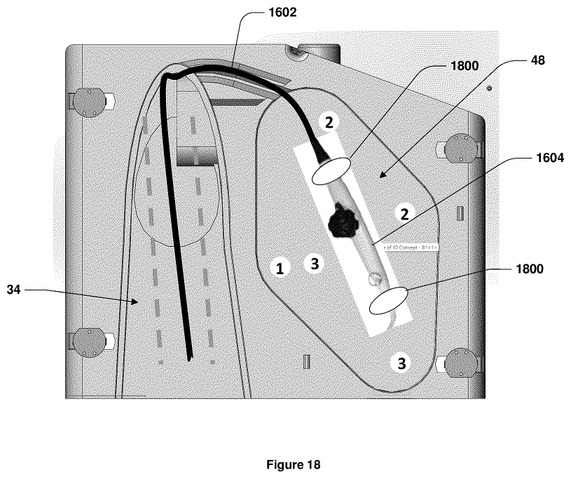

Referring to FIG. 18, the system may include a securing mechanism 1800 configured to secure a probe control body 1604 within the cord receiving cavity 48. As such, the securing mechanism 1800 may be configured to hold the TEE probe control body 1604 while the whip 1602 extends through the transition cavities into a smaller (e.g., a reduced volume) probe receiving cavity 34 containing only a distal end (i.e., a patient-exposed portion) of the whip 1602.

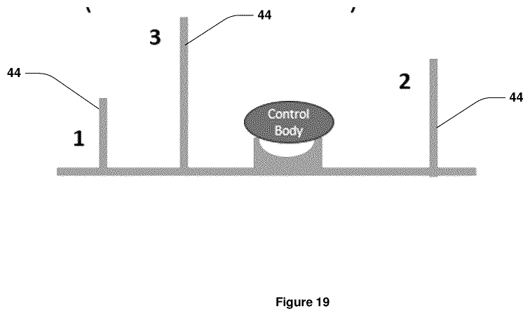

Referring to FIG. 19, a schematic cord receiving cavity cross-section is shown for reference to accentuate the elevation differences that the cord support members 44 promote for the cord 3 and potentially for the whip 1602. FIG. 19 also illustrates the location of the control body 1604 within the cord receiving cavity 48.

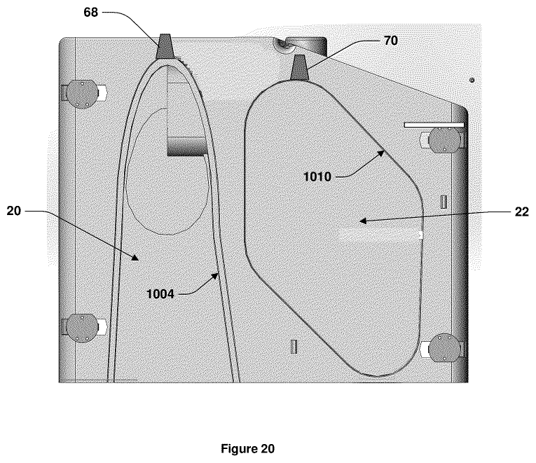

Referring to FIG. 20, a first chamber portion 1004 is defined by the housing, the first chamber portion 1004 having a first receiving cavity 20 configured to house a first medical device. A second chamber portion 1010 is defined by the housing, the second chamber portion 1010 having a second receiving cavity 22 configured to house a second medical device. The first receiving cavity 20 being distinct from the second receiving cavity 22. The disinfecting chamber includes one or more disinfecting portions in operable communication with the first chamber portion and one or more disinfecting portions in operable communication with the second chamber portion. As with previous examples, the disinfectant is in operable communication with the one or more disinfecting portions. The one or more disinfecting portions introduce the disinfectant within the first chamber portion to disinfect the first medical device. Similarly, the one or more disinfecting portions introduce the disinfectant within the second chamber portion to disinfect the second medical device. A set of defining surfaces of the first chamber portion and second chamber portion prevent passage of the disinfectant between the first chamber portion and the second chamber portion. In other words, the first chamber portion and the second chamber portion can be separated to disinfect multiple medical devices at a single time.

In this arrangement, as in some other previously described arrangements, a number of types of the probe (or other medical devices) can be secured within and disinfected in either the probe receiving cavity or the cord receiving cavity. Additionally, one or more disinfecting portions in operable communication with the probe chamber portion and the cord chamber portion can produce a first level of disinfection in the probe chamber portion and a second level of disinfection in the cord chamber portion.

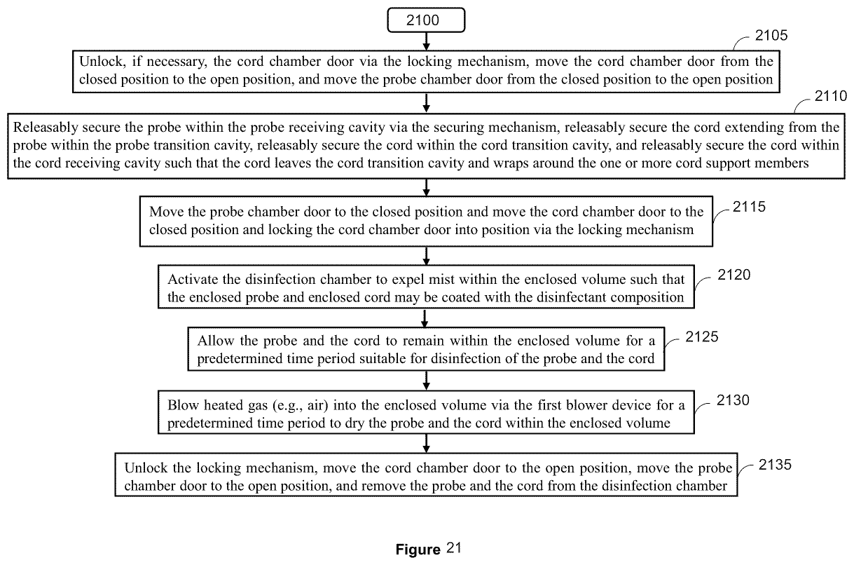

FIG. 21 illustrates a flow diagram for an exemplary method for disinfecting a probe 1 and its associated cord 3. At 2105, the method 2100 may include unlocking, if necessary, the cord chamber door via the locking mechanism, moving the cord chamber door from the closed position to the open position, and moving the probe chamber door from the closed position to the open position. At 2110, the method 2100 may include releasably securing the probe within the probe receiving cavity via the securing mechanism, releasably securing a portion of the cord extending from the probe within the probe transition cavity, releasably securing a portion of the cord within the cord transition cavity, and releasably securing a portion of the cord within the cord receiving cavity such that the cord passes from the cord transition cavity and wraps around the one or more cord support members. In some implementations, the cord may be wrapped around the one or more cord support members in an ordered manner, such as, for example, around the first cord support member, the second cord support member, the third cord support member, the fourth cord support member, the fifth cord support member, the sixth support cord member, the seventh support cord member, the eighth support cord member, the ninth support cord member, and, finally, around the tenth cord support member.

At 2115, the method 2100 may include moving the probe chamber door to the closed position to form an enclosed volume within the probe chamber portion (i.e., the probe may be enclosed between the probe receiving cavity and the enclosing portion of the probe chamber door and a portion of the cord extending from the probe may be enclosed between the probe transition cavity and the enclosing portion of the probe chamber door), and moving the cord chamber door to the closed position (locking the cord chamber door into position via the locking mechanism) to form an enclosed volume within the cord chamber portion (i.e., a portion of the cord extending from the probe transition cavity may be enclosed between the cord transition cavity and the enclosing portion of the cord chamber door, the portion of the cord wrapped around the one or more cord support members may be enclosed between the cord receiving cavity and the enclosing portion of the cord chamber door, and a remaining portion of the cord may pass through the exit aperture and out of the enclosed space).

At 2120, the method 2100 may include activating the disinfection chamber to introduce disinfectant within the enclosed volumes such that the enclosed probe and enclosed cord portions may be exposed to and/or coated with the disinfectant composition. In some implementations, at least one of the probe, the cord, or the whip have exterior surfaces that are completely (e.g., 100%) coated with the disinfectant composition. In some implementations, the term “coated” can include other disinfectant composition coverage of the probe, the cord, and the whip, including, but not limited to: substantially completely coated (e.g., 90% to 100%), 80% to 90% coated, completely coated except for surface areas in contact with support structures, coated to satisfy a particular disinfection specification for a particular device, etc.

For example, a user may activate the disinfection chamber via the display (e.g., a touch screen display) to generate the droplets of disinfectant composition (e.g., electrolyzed water), via the probe disinfecting portion and the cord disinfection portion, and to expel the generated droplets as a mist (i.e., the droplets may have a size in the range of twelve microns to forty microns) within the enclosed volume such that the probe and cord may be coated with the mist. In some implementations, the droplets may have a size (e.g. an average diameter) that is greater than forty microns. It is to be understood that the probe disinfecting portion and the cord disinfecting portion can be configured to introduce the disinfectant composition in any suitable droplet size or flow pattern other than a mist. For example, the probe disinfecting portion and the cord disinfecting portion can be configured to produce a spray-like stream, a shower-like stream, or even an uninterrupted, continuous stream of disinfectant solution while also considering the benefits of reducing a consumption volume or an introduction rate of the disinfectant composition.

The probe disinfecting portion and the cord disinfecting portion may introduce the disinfection composition (e.g., expel the mist) within the enclosed volumes such that the enclosed probe and the enclosed portions of the cord or a whip may be coated with the mist for a predetermined time period, such as, for example, eight minutes, ten minutes, or any other suitable period of time allowing disinfection or sterilization of the probe and portions of the cord. The probe disinfecting portion and the cord disinfecting portion may expel the mist within the enclosed volumes continuously, periodically, or in any other suitable manner. Some implementations can include no expelling of mist into at least one of the enclosed volumes. For example, a particular disinfection specification for a particular device may include disinfection of the probe in one enclosed volume but no disinfection of the cord in the other enclosed volume. Similarly, the predetermined time period of disinfectant introduction into one enclosed volume can differ from the predetermined time for another enclosed volume.

The probe disinfecting portion can also be of a different type than the cord disinfecting portion to effectively introduce the disinfection composition in different spray or coating patterns. In some implementations, the differing disinfecting portions can be used to effectively introduce two or more different disinfection compositions into two or more enclosed volumes as desired.

At 2125, the method 2100 may allow the probe and the cord to remain within the enclosed volume for a predetermined time period suitable for disinfection of the probe and the cord. At 2130, after the predetermined time period has elapsed, the method 2100 may blow heated gas (e.g., air with no disinfectant composition) into the enclosed volume via the heated blower for a predetermined time period to dry the probe and the cord within the enclosed volume. At 2135, the method 2100 may unlock the locking mechanism, move the cord chamber door to the open position, move the probe chamber door to the open position, and remove the probe and the cord from the disinfection chamber.

### Definitions

The following includes definitions of selected terms employed herein. The definitions include various examples or forms of components that fall within the scope of a term and that may be used for implementation. The examples are not intended to be limiting. Both singular and plural forms of terms may be within the definitions.

As used herein, an “operable connection” or “operable coupling,” or a connection by which entities are “operably connected” or “operably coupled” is one in which the entities are connected in such a way that the entities may perform as intended. An operable connection may be a direct connection or an indirect connection in which an intermediate entity or entities cooperate or otherwise are part of the connection or are in between the operably connected entities. In the context of signals, an “operable connection,” or a connection by which entities are “operably connected,” is one in which signals, physical communications, or logical communications may be sent or received. Typically, an operable connection includes a physical interface, an electrical interface, or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical or physical communication channels can be used to create an operable connection.

While example systems, methods, and so on, have been illustrated by describing examples, and while the examples have been described in considerable detail, it is not the intention of the applicants to restrict or in any way limit scope to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

What is claimed is:

1. A disinfection chamber for disinfecting a medical probe including a probe cord, comprising:
a housing;
a probe chamber portion defined by the housing, the probe chamber portion having a probe receiving cavity configured to house the medical probe;
a cord chamber portion defined by the housing, the cord chamber portion having a cord receiving cavity configured to house a majority of the probe cord of the medical probe, the cord receiving cavity being distinct from the probe receiving cavity;
one or more disinfecting portions in operable communication with the probe chamber portion and the cord chamber portion;
a disinfectant in operable communication with the one or more disinfecting portions; wherein the one or more disinfecting portions introduce the disinfectant within the probe chamber portion and the cord chamber portion to disinfect the probe and a majority of the probe cord surface;
a probe transition cavity; and
a cord transition cavity,
wherein the probe transition cavity and the cord transition cavity define a passageway between the probe receiving cavity and the cord receiving cavity, and
wherein the one or more disinfecting portions include a nozzle and an electrostatically charged diode configured to impart an electrostatic charge to the disinfectant.

2. A disinfection chamber for disinfecting a medical probe including a probe cord, comprising:
a housing including a probe chamber surface, a probe chamber side wall, a cord chamber surface, and a cord chamber side wall;
a probe chamber portion defined by the probe chamber surface and the probe chamber side wall of the housing, the probe chamber portion having a probe receiving cavity configured to house the medical probe;
a cord chamber portion defined by the cord chamber surface and the cord chamber side wall of the housing, the cord chamber portion having a cord receiving cavity configured to house a majority of the probe cord of the medical probe, the cord receiving cavity being distinct from the probe receiving cavity;
one or more disinfecting portions in operable communication with the probe chamber portion and the cord chamber portion through at least one of an aperture in the probe chamber side wall or an aperture in the cord chamber side wall;
a disinfectant in operable communication with the one or more disinfecting portions; wherein the one or more disinfecting portions introduce the disinfectant within the probe chamber portion and the cord chamber portion to disinfect the probe and a majority of the probe cord surface;
a probe transition cavity; and
a cord transition cavity,
wherein the probe transition cavity and the cord transition cavity define a passageway between the probe receiving cavity and the cord receiving cavity.

3. The disinfection chamber of claim 2, wherein:
the probe chamber side wall includes a probe chamber bottom wall,
the probe chamber bottom wall defines a bottom wall aperture configured to place the one or more disinfecting portions in operable communication with the probe receiving cavity,
the cord chamber side wall includes a cord chamber bottom wall,
the cord chamber bottom wall defines a bottom wall aperture configured to place the one or more disinfecting portions in operable communication with the cord receiving cavity.

4. The disinfection chamber of claim 3, wherein the probe chamber bottom wall and the cord chamber bottom wall are configured to direct a quantity of the disinfectant toward the one or more disinfecting portions.

5. The disinfection chamber of claim 2, wherein the probe chamber side wall defines a side wall aperture configured to place the one or more disinfecting portions in operable communication with probe receiving cavity such that the disinfectant is introduced in a direction parallel or substantially parallel to the probe chamber surface.

6. The disinfection chamber of claim 2, wherein the cord chamber side wall defines a side wall aperture configured to place the one or more disinfecting portions in operable communication with the cord receiving cavity such that the disinfectant is introduced in a direction parallel or substantially parallel to the cord chamber surface.

7. The disinfection chamber of claim 2, wherein the one or more disinfecting portions includes one or more nozzles such that:
a first nozzle of the one or more disinfecting portions is in operable communication with the probe receiving cavity,
a second nozzle of the one or more disinfecting portions is in operable communication with the cord receiving cavity, and
the first nozzle is configured to operate independently of the second nozzle in at least one of:
a size of a droplet of the disinfectant produced,
a volume of the disinfectant introduced into one of the probe receiving cavity or the cord receiving cavity, or
a type of nozzle.

8. The disinfection chamber of claim 2, further comprising:
a disinfectant storage container attached to or contained within the housing, the disinfectant storage container configured to contain the disinfectant; and
at least one of a conductivity sensor or an electrochemical sensor attached to the housing, the sensor configured to measure at least one of a conductivity property or an electrochemical property of the disinfectant.

9. The disinfection chamber of claim 2, further comprising:
a first cord support member attached to and extending away from the cord chamber surface; and
a second cord support member attached to and extending away from the cord chamber surface, wherein:
the first cord support member supports the cord a first distance from the cord chamber surface,
the second cord support member supports the cord at a second distance from the cord chamber surface, and
the first distance is different from the second distance such that a portion of the cord can be maintained at the first distance from the cord chamber surface and a first end of the cord and a second end of the cord can be maintained at the second distance from the cord chamber surface.

10. The disinfection chamber of claim 9, wherein the cord chamber surface defines a convex portion extending toward a cord chamber door such that the first cord support member having a first length is configured to support the cord at the first distance from the cord chamber surface and the second cord support member having the first length is configured to support the cord at the second distance from the cord chamber surface, wherein the convex portion of the cord chamber surface is configured to minimize a volume of the cord receiving cavity.

11. The disinfection chamber of claim 9, further comprising a sensor attached to the first cord support member, the sensor configured to sense contact between the cord and the first cord support member.

12. The disinfection chamber of claim 2, further comprising a whip support or multiple whip supports attached to and extending away from the probe chamber surface, the whip support configured to support a probe whip a distance away from the probe chamber surface.

13. The disinfection chamber of claim 2, further comprising:
a second probe transition cavity; and
a second cord transition cavity,
wherein the second probe transition cavity and the second cord transition cavity define a second passageway between the probe receiving cavity and the cord receiving cavity, the second passageway enables a probe whip attached to the probe to pass from the probe receiving cavity to the cord receiving cavity such that a portion of the probe whip is received within the cord receiving cavity.

14. The disinfection chamber of claim 2, wherein:
the cord chamber surface includes a securing mechanism configured to secure a probe within the cord receiving cavity, and
the probe whip extends through a transition chamber into the probe receiving cavity.

15. The disinfection chamber of claim 14, wherein the one or more disinfecting portions includes one or more nozzles such that:
a first nozzle of the one or more disinfecting portions is in operable communication with the probe receiving cavity,
a second nozzle of the one or more disinfecting portions is in operable communication with the cord receiving cavity, and
the first nozzle is configured to operate independently of the second nozzle in at least one of:
a size of a droplet of the disinfectant produced,
a volume of the disinfectant introduced into one of the probe receiving cavity or the cord receiving cavity, or
a type of nozzle.

16. The disinfection chamber of claim 2, further comprising a radio frequency identification (RFID) system to create a searchable database including disinfection information relating to the probe, the RFID system comprising:
an RFID tag unique to the probe;
an RFID tag unique to a container of disinfectant;
a unique identity for a user of the RFID system; and
an associated RFID reader configured to communicate with a controller and a programmable logic controller.

17. The disinfection chamber of claim 2, further comprising:
a securing mechanism attached to the probe chamber surface and extending into the probe receiving cavity, the securing mechanism configured to support a probe within the probe receiving cavity;
a second securing mechanism attached to the cord chamber surface and extending into the cord receiving cavity, the second securing mechanism configured to support a probe within the cord receiving cavity; and
a cord support structure attached to the cord chamber surface and extending into the cord receiving cavity, the cord support structure configured to support at least one of a cord or a whip within the cord receiving cavity, such that:
a number of types of the probe can be secured within and disinfected in either the probe receiving cavity or the cord receiving cavity, and
one or more disinfecting portions in operable communication with the probe chamber portion and the cord chamber portion can produce a first level of disinfection in the probe chamber portion and a second level of disinfection in the cord chamber portion, and
the passageway between the probe receiving cavity and the cord receiving cavity is located at least one of at or near a bottom of each of the probe receiving cavity and the cord receiving cavity.

18. The disinfection chamber of claim 2, further comprising a probe chamber door configured to cooperate with the probe chamber surface to define the probe receiving cavity, the probe chamber door having a generally flat face cooperating with the probe chamber surface such that the generally flat face of the probe chamber door does not complement a shape profile of the probe receiving cavity.

19. The disinfection chamber of claim 2, wherein the one or more disinfecting portions is configured to introduce the disinfectant within the probe chamber portion and the cord chamber portion using at least one of a moving gas or a hydraulic fluid.

20. The disinfection chamber of claim 2, wherein the disinfecting portion includes at least one of a nozzle or a perforated surface to introduce the disinfectant within the probe chamber portion and the cord chamber portion.

21. The disinfection chamber of claim 2, further comprising a flexible structure in operable communication with at least one of the probe chamber portion or the cord chamber portion to accommodate a pressure change within at least one of the probe chamber portion or the cord chamber portion.

22. The disinfection chamber of claim 2, further comprising a flexible structure configured to expand and contract to accommodate pressure changes within both the probe chamber portion and the cord chamber portion.

23. The disinfecting chamber of claim 2, further comprising a flexible structure that is formed into a wall of at least one of the probe chamber portion or the cord chamber portion.

24. A disinfection chamber for disinfecting one or more reusable medical devices, comprising:
a housing;
a first chamber portion defined by the housing, the first chamber portion having a first receiving cavity configured to house a medical device;
a second chamber portion defined by the housing, the first receiving cavity being distinct from the second receiving cavity;
one or more disinfecting portions in operable communication with the first chamber portion;
one or more disinfecting portions in operable communication with the second chamber portion;
a disinfectant in operable communication with the one or more disinfecting portions; wherein:
the one or more disinfecting portions introduce the disinfectant within the first chamber portion to disinfect the medical device,
the one or more disinfecting portions introduce the disinfectant within the second chamber portion, and
a set of defining surfaces of the first chamber portion and second chamber portion prevent passage of the disinfectant between the first chamber portion and the second chamber portion;
a conductivity sensor attached to the housing, the conductivity sensor configured to measure a conductivity property of the disinfectant; and
an identification system to create a searchable database including disinfection information relating to the probe.

25. A disinfection chamber for disinfecting one or more reusable medical devices, comprising:
a housing;
a first chamber portion defined by the housing, the first chamber portion having a first receiving cavity configured to house a first medical device;
a second chamber portion defined by the housing, the second chamber portion having a second receiving cavity configured to house a second medical device, the first receiving cavity being distinct from the second receiving cavity;
one or more disinfecting portions in operable communication with the first chamber portion;
one or more disinfecting portions in operable communication with the second chamber portion;
a disinfectant in operable communication with the one or more disinfecting portions; wherein:
the one or more disinfecting portions introduce the disinfectant within the first chamber portion to disinfect the first medical device, and
the one or more disinfecting portions introduce the disinfectant within the second chamber portion to disinfect the second medical device, a set of defining surfaces of the first chamber portion and second chamber portion prevent passage of the disinfectant between the first chamber portion and the second chamber portion;
a conductivity sensor attached to the housing, the conductivity sensor configured to measure a conductivity property of the disinfectant; and
an identification system to create a searchable database including disinfection information relating to the probe.

26. The disinfection chamber of claim 25, wherein the one or more disinfecting portions includes one or more nozzles such that:
a first nozzle of the one or more disinfecting portions is in operable communication with the first receiving cavity,
a second nozzle of the one or more disinfecting portions is in operable communication with the second receiving cavity, and
the first nozzle is configured to operate independently of the second nozzle in at least one of:
a size of a droplet of the disinfectant produced,
a volume of the disinfectant introduced into one of the first receiving cavity or the second receiving cavity,
a type of the disinfectant, or
a type of the nozzle.

27. The disinfection chamber of claim 25, further comprising:
a first chamber door attached to the housing, the first chamber door configured to selectively enclose the first receiving cavity;
a second chamber door attached to the housing, the second chamber door configured to selectively enclose the second receiving cavity;
a first locking mechanism attached to the first chamber door; and
a second locking mechanism attached to the second chamber door, the first locking mechanism and the second locking mechanism are configured to operate dependently such that only one of the first chamber door and the second chamber door are operable at one time.

28. The disinfection chamber of claim 25, wherein the identification includes RFID technology.
